(19) United States
(12) Patent Application Publication
(10) Pub. No.: US 2026/0272426 A1
(43) Pub. Date: Sep. 17, 2026

(54) MEDICAL IMAGE PROCESSING DEVICE

(71) Applicant: CANON MEDICAL SYSTEMS CORPORATION, Otawara-shi (JP)

(72) Inventors: Masaki YOSHIDA, Yaita (JP); Koji ANDO, Otawara (JP); Yoshihito ABE, Utsunomiya (JP); Kazuyo SAITO, Nasushiobara (JP); Hiroki TAWARA, Bunkyo (JP); Kentaro SYOJI, Nasushiobara (JP); Nobuo OGURA, Otawara (JP); Satoshi KAWAGUCHI, Kawasaki (JP)

(73) Assignee: CANON MEDICAL SYSTEMS CORPORATION, Otawara-shi (JP)

(21) Appl. No.: 19/565,038

(22) Filed: Mar. 12, 2026

(30) Foreign Application Priority Data
Mar. 17, 2025 (JP) 2025-042659

(51) Int. Cl. A61B 8/00; A61B 6/00; A61B 8/06; G06T 7/00; G06T 7/70

(52) U.S. Cl. CPC A61B 8/5261; A61B 8/065; A61B 8/463; A61B 8/488; G06T 7/0012; G06T 7/70; A61B 6/5247; G06T 2207/10024; G06T 2207/10081; G06T 2207/10088; G06T 2207/10116; G06T 2207/10132; G06T 2207/30048; G06T 2207/30104

(57) ABSTRACT

A medical image processing device according to an embodiment includes a position acquirer, a blood flow information acquirer, and a display controller. The position acquirer acquires a position of a target site on a medical image. The blood flow information acquirer acquires blood flow information of a first region in a periphery of the target site. The display controller displays an image indicating a relationship of the blood flow information acquired by the blood flow information acquirer relative to the position of the target site acquired by the position acquirer on the target site or on a second region that differs from the first region.

### CROSS REFERENCE TO RELATED APPLICATIONS

This application is based upon and claims the benefit of priority from the prior Japanese Patent Application No. 2025-42659, filed on Mar. 17, 2025, the entire contents of which are incorporated herein by reference.

### FIELD

The embodiments disclosed in the present specification and in the drawings relate to a medical image processing device.

### BACKGROUND

Transcatheter mitral valve repair is known as one of the catheter-based procedures performed for mitral valve regurgitation (MR), a disease of the heart's mitral valve. In transcatheter mitral valve repair, a clip called the Mitra Clip is placed at the site of the mitral valve where regurgitation is occurring, thereby reducing the regurgitation.

When performing transcatheter mitral valve repair, the surgeon confirms information on the regurgitation site of the mitral valve based on a color Doppler image. Then, based on the confirmed information, the surgeon plans where the clip is to be placed and assesses blood flow after clip placement.

However, since color Doppler images display the regurgitation site in color, it is difficult to visually recognize the shape of the mitral valve itself. Since the shape of the mitral valve itself is difficult to visually recognize, it is difficult to determine at which position and in which direction the clip should be placed.

### BRIEF DESCRIPTION OF THE DRAWINGS

FIG. 1 is a block diagram showing a configuration example of a medical image processing device according to an embodiment;

FIG. 2 is a diagram showing transcatheter mitral valve repair to which the medical image processing device according to the embodiment can be applied;

FIG. 3 is a flow chart showing an operation example of the medical image processing device according to the embodiment;

FIG. 4 is a diagram showing an acquisition step of a medical image in the operation example of the medical image processing device according to the embodiment;

FIG. 5 is a diagram showing a generation step of correspondence information in the operation example of the medical image processing device according to the embodiment;

FIG. 6 is a diagram showing a display step of a relationship image in the operation example of the medical image processing device according to the embodiment;

FIG. 7 is a diagram showing a display step of a relationship image in an operation example of a medical image processing device according to a first modification of the embodiment;

FIG. 8 is a diagram showing a display step of a relationship image in an operation example of a medical image processing device according to a second modification of the embodiment;

FIG. 9 is a diagram showing a display step of a relationship image in an operation example of a medical image processing device according to a third modification of the embodiment;

FIG. 10 is a diagram showing a display step of a relationship image in an operation example of a medical image processing device according to a fourth modification of the embodiment;

FIG. 11 is a diagram showing a display step of a relationship image in an operation example of a medical image processing device according to a fifth modification of the embodiment;

FIG. 12 is a diagram showing a display step of a relationship image in an operation example of a medical image processing device according to a sixth modification of the embodiment;

FIG. 13 is a diagram showing a display step of a relationship image in an operation example of a medical image processing device according to a seventh modification of the embodiment;

FIG. 14 is a diagram showing a display step of a relationship image in an operation example of a medical image processing device according to an eighth modification of the embodiment;

FIG. 15 is a diagram showing a display step of a relationship image in an operation example of a medical image processing device according to a ninth modification of the embodiment; and

FIG. 16 is a flow chart showing an operation example of the medical image processing device according to the embodiment.

### DETAILED DESCRIPTION

Hereinafter, an embodiment of a medical image processing device will be described with reference to the drawings. In the following description, constituent elements with substantially a same function and a same configuration will be denoted by a same reference sign and overlapping descriptions will only be given when necessary.

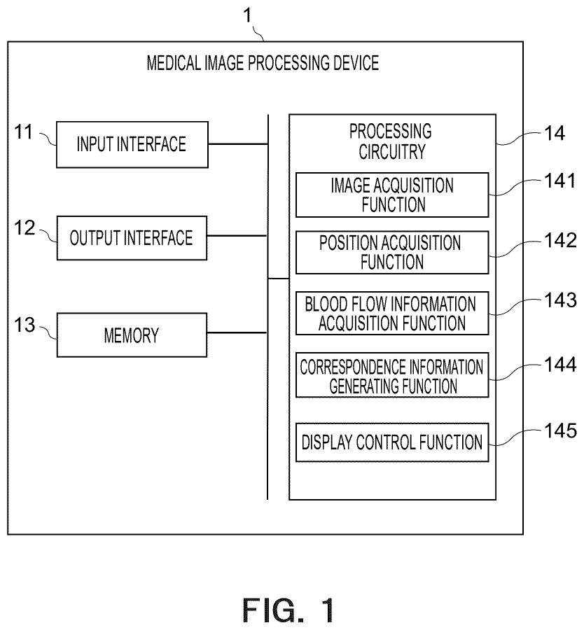

FIG. 1 is a block diagram showing a configuration example of a medical image processing device 1 according to the embodiment. For example, the medical image processing device 1 is incorporated into medical image diagnostic devices such as ultrasound diagnostic devices, X-ray diagnostic devices, X-ray CT devices, and magnetic resonance imaging (MRI) devices.

As shown in FIG. 1, the medical image processing device 1 includes an input interface 11, an output interface 12, a memory 13, and processing circuitry 14. The input interface 11, the output interface 12, and the memory 13 are communicably connected to the processing circuitry 14.

The input interface 11 accepts various input operations from an operator. The input interface 11 converts the accepted input operations into electric signals, and outputs the electric signals to the processing circuitry 14. For example, the input interface 11 accepts an input operation for displaying a relationship image showing a relationship of blood flow information relative to a position of a target site from the operator. Details of the relationship image will be provided later. As the input interface 11, for example, a mouse, a keyboard, a trackball, a switch, a button, a joystick, a touch pad, or a touch panel display can be used as appropriate.

The output interface 12 outputs various kinds of information. For example, the output interface 12 includes a display. The display outputs the relationship image generated by the processing circuitry 14, graphical user interfaces (GUIs) for accepting various operations from the operator, and the like. For example, a liquid crystal display (LCD), a cathode ray tube (CRT) display, an organic electro luminescence display (OELD), a plasma display, or any other display can be used as appropriate as the display. The output interface 12 may include a speaker.

The memory 13 is a non-transitory storage device that stores various kinds of information, such as a hard disk drive (HDD), an optical disk, a solid-state drive (SSD), and an integrated circuit memory device. For example, the memory 13 stores a control program for controlling the medical image processing device 1 and various kinds of data used to execute the control program. In addition to an HDD and an SSD, the memory 13 may also be a drive device that writes and reads various kinds of information to and from portable storage media such as a compact disc (CD), a digital versatile disc (DVD), and a flash memory or semiconductor memory devices such as a random access memory (RAM). Note that the memory 13 need not necessarily be realized by a single storage device. For example, the memory 13 may be realized by a plurality of storage devices.

The processing circuitry 14 is circuitry that controls operations of the medical image processing device 1 as a whole in response to electrical signals of input operations input from the input interface 11. For example, the processing circuitry 14 includes an image acquisition function 141, a position acquisition function 142, a blood flow information acquisition function 143, a correspondence information generating function 144, and a display control function 145.

In this case, for example, each of the processing functions executed by the image acquisition function 141, the position acquisition function 142, the blood flow information acquisition function 143, the correspondence information generating function 144, and the display control function 145 which are constituent elements of the processing circuitry 14 shown in FIG. 1 is recorded in the memory 13 in a computer-executable form. The processing circuitry 14 is, for example, a processor. The processor that constitutes the processing circuitry 14 reads each program from the memory 13 and realizes a function corresponding to each read program by executing the program. In other words, the processing circuitry 14 in a state of having read each program is to include each function shown in the processing circuitry 14 in FIG. 1.

While FIG. 1 shows a case where the image acquisition function 141, the position acquisition function 142, the blood flow information acquisition function 143, the correspondence information generating function 144, and the display control function 145 are realized by the single processing circuitry 14, the embodiment is not limited thereto. For example, the processing circuitry 14 may be configured as a combination of a plurality of independent processors which realizes each processing function by having each processor execute each program. In addition, each processing function included in the processing circuitry 14 may be realized by being appropriately integrated to a single processing circuitry or distributed among a plurality of processing circuitries.

The image acquisition function 141 acquires a medical image of a subject. For example, the medical image is an ultrasound image, an X-ray image, a computed tomography (CT) image, a magnetic resonance (MR) image, and the like. The image acquisition function 141 acquires the medical image by, for example, generating the medical image by itself based on inspection data (for example, ultrasonic waves, X-rays, electromagnetic waves, and the like) obtained from the subject. The image acquisition function 141 may acquire, via communication or a portable memory, images generated by a modality that differs from the medical image diagnostic device in which the medical image processing device 1 is incorporated. The image acquisition function 141 may generate and acquire a medical image during a procedure. Alternatively, the image acquisition function 141 may read and acquire, from the memory 13, a medical image generated and recorded in the memory 13 at a specific time point in the past. Alternatively, the image acquisition function 141 may read and acquire, from the memory 13, a medical image generated and recorded in the memory 13 within a specific time range in the past.

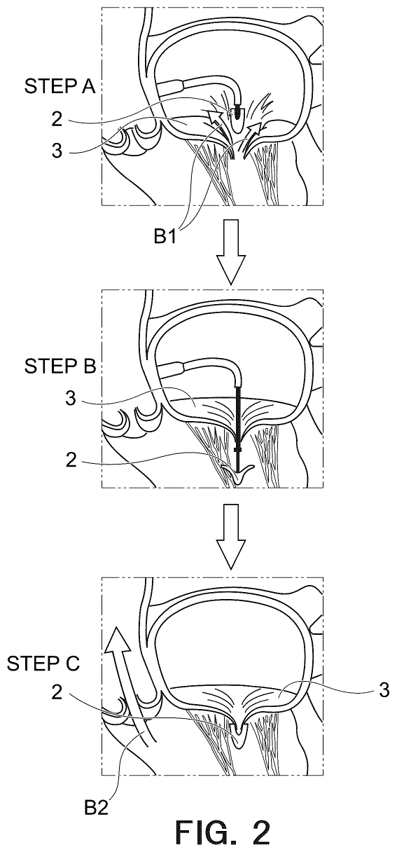

The position acquisition function 142 acquires a position of a target site on the medical image acquired by the image acquisition function 141. For example, the position acquisition function 142 performs region extraction processing such as segmentation processing and acquires the position of the target site based on the result of the region extraction processing. FIG. 2 is a diagram showing transcatheter mitral valve repair to which the medical image processing device 1 according to the embodiment can be applied. In the example shown in FIG. 2, the target site is a mitral valve 3 of the subject that is an example of a procedure object site. As shown in FIG. 2, in transcatheter mitral valve repair, a clip 2 attached to the tip of a catheter that is an example of a procedure device is inserted through a vein at the subject's groin and advanced from the right atrium to the left atrium (STEP A). Next, the clip 2 is moved to a site of the mitral valve 3 where regurgitation B1 is most prominent, the clip 2 is opened, and the mitral valve 3 is placed on the opened clip 2. After the mitral valve 3 is placed on the clip 2, the clip 2 is closed (STEP B). Once the clip 2 is closed, the catheter is extracted and the clip 2 is fixed to the mitral valve 3 (STEP C). By fixing the clip 2 to the mitral valve 3, the regurgitation B1 can be suppressed and blood flow B2 can be improved so it flows from the left ventricle toward the vena cava. In the example shown in FIG. 2, the position acquisition function 142 acquires the position of the mitral valve 3 based on an image of the heart of the subject acquired by the image acquisition function 141.

The blood flow information acquisition function 143 shown in FIG. 1 acquires blood flow information of a first region in a periphery of the target site. Blood flow information is information related to blood flow. For example, blood flow information includes a direction of blood flow, a blood flow amount, and a blood flow velocity. The blood flow information acquisition function 143 may acquire blood flow information in real time during procedures. In other words, based on a medical image generated in real time during a procedure by the image acquisition function 141, the blood flow information acquisition function 143 may acquire blood flow information in the medical image. Alternatively, the blood flow information acquisition function 143 may acquire blood flow information at a specific time point in the past. In other words, based on a medical image generated at a specific time point in the past by the image acquisition function 141 and stored in the memory 13, the blood flow information acquisition function 143 may acquire blood flow information in the medical image. Alternatively, the blood flow information acquisition function 143 may acquire blood flow information within a specific time range in the past. In other words, based on a medical image generated within a specific time range in the past by the image acquisition function 141 and stored in the memory 13, the blood flow information acquisition function 143 may acquire blood flow information in the medical image.

The blood flow information acquisition function 143 may acquire blood flow information based on at least one of a color Doppler image as a medical image and changes in the shape of the heart or blood vessels in the medical image. In addition, the blood flow information acquisition function 143 may update the acquisition of blood flow information in accordance with progress of procedures.

The correspondence information generating function 144 generates correspondence information indicating a correspondence relationship between a plurality of positions on the target site and blood flow information. The correspondence information generating function 144 may generate the correspondence information by associating a plurality of positions on the target site with blood flow information existing in a specific direction relative to each of the plurality of positions. The specific direction may include at least one of a direction of blood flow, a normal direction of a surface of the target site, a travel direction of the procedure device, and a scan line direction of ultrasound in a region of interest.

The display control function 145 displays a relationship image indicating a relationship of the blood flow information acquired by the blood flow information acquisition function 143 relative to the position of the target site acquired by the position acquisition function 142 on the target site or on a second region that differs from the first region. The second region may be a region excluding the region through which the procedure device has passed and the region through which the procedure device is to pass. The second region can also be referred to as a region that does not impair visibility of the target site. The display control function 145 displays the relationship image via a display. Based on the correspondence information generated by the correspondence information generating function 144, the display control function 145 displays the relationship image so as to indicate a relationship corresponding to each of the plurality of positions on the target site.

The relationships indicated by the relationship image may include a direction of blood flow in the blood flow information relative to the positions of the target site. In this case, the direction of blood flow may be a direction that differs from the normal direction of blood flow. In this case, the direction that differs from the normal direction may be a direction opposite to the normal direction. The relationships indicated by the relationship image may further include at least one of an amount of blood flow in the direction of blood flow and a blood flow velocity in the direction of blood flow.

The relationship image may include at least one of a color map, a color bar, a color histogram, an arrow, and a line segment.

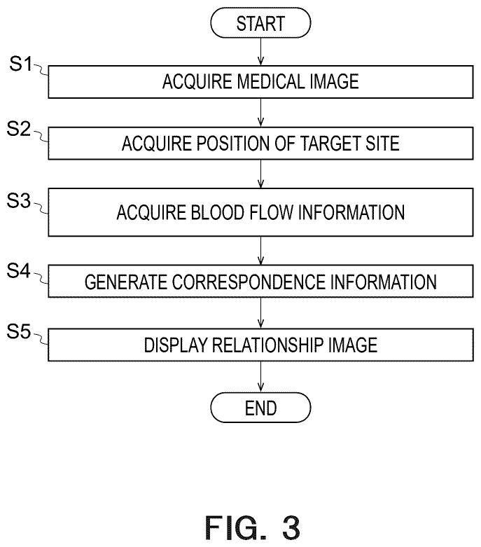

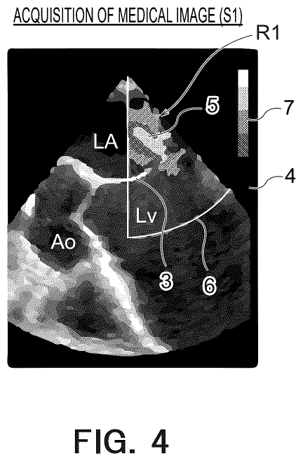

Next, an operation example of the medical image processing device 1 according to the embodiment configured as described above will be described. FIG. 3 is a flow chart showing an operation example of the medical image processing device 1 according to the embodiment. First, as shown in FIG. 3, the image acquisition function 141 acquires a medical image (step S1). FIG. 4 is a diagram showing an acquisition step of a medical image in the operation example of the medical image processing device 1 according to the embodiment. In the example shown in FIG. 4, the image acquisition function 141 acquires an ultrasound image 4 that is an example of a medical image. More specifically, the image acquisition function 141 acquires a color Doppler image created by superimposing a color blood flow image 5 indicating blood flow information on a black-and-white B-mode image as the ultrasound image 4. In FIG. 4, reference sign LA denotes the left atrium. Reference sign LV denotes the left ventricle. Reference sign Ao denotes the aorta. Reference sign 6 denotes a region of interest. In the example shown in FIG. 4, the blood flow image 5 is displayed in the first region in the periphery of the mitral valve 3. In addition, the blood flow image 5 has a corresponding color within a color gauge 7 that indicates a color for each blood flow velocity. In addition, in the example shown in FIG. 4, the blood flow image 5 represents a regurgitant component flowing from the left ventricle LV into the left atrium LA.

Here, the medical image processing device 1 may be incorporated into an ultrasound diagnostic device and the image acquisition function 141 may generate and acquire a medical image by itself. In this case, an ultrasound probe (not illustrated) is connected via transmission/reception circuitry (not illustrated) to the processing circuitry 14. The image acquisition function 141 of the processing circuitry 14 generates the ultrasound image 4 in response to scanning of the subject by an ultrasound probe. More specifically, for example, the image acquisition function 141 receives reflected wave data from the transmission/reception circuitry, performs logarithmic amplification, envelope detection processing, and the like, and generates data (B-mode data) where signal intensity is expressed in terms of brightness. In addition, the image acquisition function 141 performs frequency analysis on velocity information from the reflected wave data received from the transmission/reception circuitry, extracts blood flow, tissue, and contrast agent echo components based on the Doppler effect, and generates data (Doppler data) representing extracted mobile object information such as velocity, dispersion, and power at multiple points. Furthermore, the image acquisition function 141 may be capable of processing both two-dimensional reflected wave data and three-dimensional reflected wave data. In other words, the image acquisition function 141 may generate two-dimensional B-mode data from two-dimensional reflected wave data and generate three-dimensional B-mode data from three-dimensional reflected wave data. In addition, the image acquisition function 141 may generate two-dimensional Doppler data from two-dimensional reflected wave data and three-dimensional Doppler data from three-dimensional reflected wave data.

In addition, the image acquisition function 141 generates a B-mode image representing an intensity of reflected waves as brightness from B-mode data. Furthermore, for example, the image acquisition function 141 generates a Doppler image that visualizes blood flow information from Doppler data. The Doppler image is velocity image data representing an average velocity of blood flow, dispersion image data representing a dispersion value of blood flow, power image data representing power of blood flow, or image data that combines these pieces of image data. In addition, as the Doppler image, the image acquisition function 141 generates a color Doppler image in which blood flow information such as the average velocity of blood flow is displayed in color, generates a power Doppler image based on the power image data, or generates a Doppler image in which one piece of blood flow information is displayed in grayscale. Furthermore, for example, the image acquisition function 141 can also generate an M-mode image from time-series data of B-mode data on one scan line. In addition, the image acquisition function 141 can also generate Doppler waveforms by plotting velocity information of blood flow and tissue over time from Doppler data.

Note that the medical image acquired in step S1 is not limited to the ultrasound image 4 and may be an X-ray image, a CT image, or an MR image.

After the medical image is acquired, as shown in FIG. 3, the position acquisition function 142 acquires a position of the target site (step S2). In the example shown in FIG. 4, the position acquisition function 142 acquires the position of the mitral valve 3.

After the position of the target site is acquired, as shown in FIG. 3, the blood flow information acquisition function 143 acquires blood flow information of the first region in the periphery of the target site (step S3). In the example shown in FIG. 4, the blood flow information acquisition function 143 acquires blood flow information of a first region R1 in the periphery of the mitral valve 3 based on the color Doppler image, in other words, the blood flow image 5. The blood flow information includes a direction of blood flow, a blood flow amount, and a blood flow velocity.

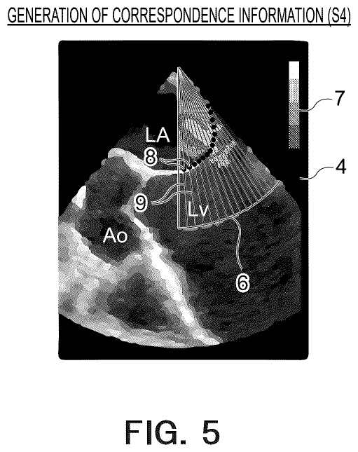

After the blood flow information is acquired, as shown in FIG. 3, the correspondence information generating function 144 generates correspondence information indicating a correspondence relationship between a plurality of positions on the target site and blood flow information (step S4). FIG. 5 is a diagram showing a generation step of correspondence information in the operation example of the medical image processing device 1 according to the embodiment. In the example shown in FIG. 5, the correspondence information generating function 144 generates the correspondence information by associating a plurality of positions 8 on the mitral valve 3 with blood flow information existing in a direction of a scan line 9 of ultrasonic waves relative to each of the plurality of positions 8. Note that the blood flow information existing on one scan line 9 that passes through one position 8 contains a plurality of blood flow velocities and a plurality of blood flow amounts. The correspondence information generating function 144 may generate the correspondence information by associating a maximum value or an average value of the plurality of blood flow velocities and the plurality of blood flow amounts with the corresponding position 8. In addition, the correspondence information generating function 144 may calculate a regurgitation amount corresponding to one position 8 based on an area, a volume, and a length of the blood flow image 5 of a portion existing on the one scan line 9 that passes through one position 8. Furthermore, the correspondence information generating function 144 may generate the correspondence information by associating the calculated regurgitation amount with the one position 8.

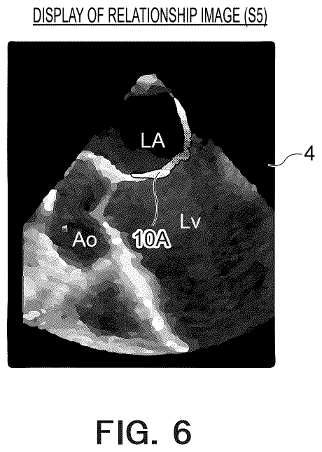

After the correspondence information is generated, as shown in FIG. 3, the display control function 145 displays a relationship image indicating a relationship of the blood flow information acquired by the blood flow information acquisition function 143 relative to the position of the target site acquired by the position acquisition function 142 on the target site or on the second region (step S5). In the example shown in FIG. 6, the display control function 145 displays a relationship image 10A on the mitral valve 3. In other words, the display control function 145 displays the relationship image 10A so as to overlap with the mitral valve 3 while conforming to the shape of the mitral valve 3. In the example shown in FIG. 6, the relationship image 10A is a color map that indicates a state of regurgitation of blood relative to the position of the mitral valve 3 using color. In the relationship image 10A, locations where the regurgitation amount and the regurgitation velocity of blood regurgitating from the left ventricle LV to the left atrium LA are greater may have more prominent colors (such as red).

According to the example shown in FIG. 6, displaying the relationship image 10A on the mitral valve 3 so as to conform to the shape of the mitral valve 3 enables a user to readily comprehend a regurgitation state based on the relationship image 10A while suppressing a decline in visibility of the mitral valve 3. In other words, the blood flow in the periphery of the mitral valve 3 can be visualized while suppressing a decline in visibility of the mitral valve 3. In addition, by displaying the relationship image 10A, procedures can be performed without having to compare a color Doppler image with an ordinary ultrasound image.

The various modifications described below can be applied to the embodiment described above.

### First Modification

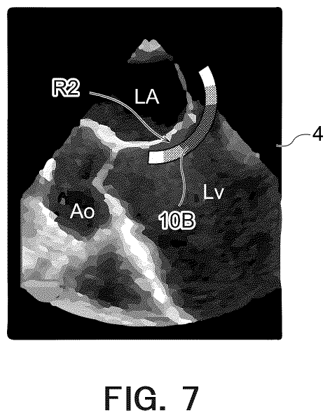

First, a first modification of displaying a relationship image on the second region R2 will be described with a focus on the differences from the embodiment described above. FIG. 7 is a diagram showing a display step of a relationship image 10B in an operation example of the medical image processing device 1 according to a first modification of the embodiment.

An example of displaying the relationship image 10A on the mitral valve 3 has been described with reference to FIG. 6. In contrast, in the example shown in FIG. 7, the display control function 145 displays the relationship image 10B on the region R2 that differs from the first region R1 (refer to FIG. 4) in which blood flow information was acquired. In the example shown in FIG. 7, the relationship image 10B is a color bar that indicates a state of regurgitation of blood relative to the position of the mitral valve 3 using color. In the relationship image 10B, locations where the regurgitation amount and the regurgitation velocity of blood regurgitating from the left ventricle LV to the left atrium LA are greater have more prominent colors (such as red).

According to the example shown in FIG. 7, displaying the relationship image 10B on the second region R2 that is separated from the mitral valve 3 enables a user to readily comprehend a regurgitation state while suppressing a decline in visibility of the mitral valve 3.

### Second Modification

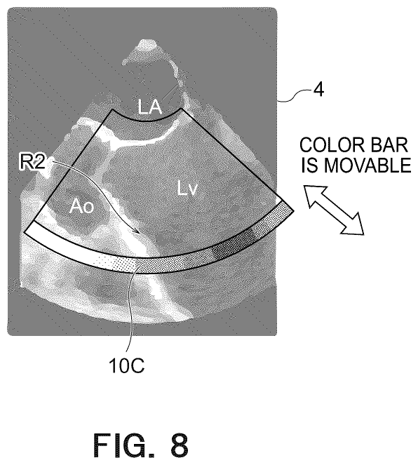

Next, a second modification of displaying a relationship image so as to be movable will be described with a focus on the differences from the embodiment described above. FIG. 8 is a diagram showing a display step of a relationship image 10C in an operation example of the medical image processing device 1 according to the second modification of the embodiment.

An example of displaying the relationship images 10A and 10B at a constant position has been described with reference to FIGS. 6 and 7. In contrast, as shown in FIG. 8, the display control function 145 displays the relationship image 10C so as to be movable in response to input operations. In the example shown in FIG. 8, the relationship image 10C is a color bar in a similar manner to FIG. 7.

Even in the example shown in FIG. 8, displaying the relationship image 10C on the second region R2 enables the user to readily comprehend a regurgitation state while suppressing a decline in visibility of the mitral valve 3. In addition, since the relationship image 10C can be moved, convenience can be improved.

### Third Modification

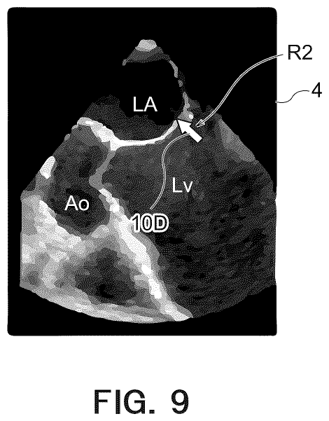

Next, a third modification of displaying an arrow as a relationship image will be described with a focus on the differences from the embodiment described above. FIG. 9 is a diagram showing a display step of a relationship image 10D in an operation example of the medical image processing device 1 according to the third modification of the embodiment.

Thus far, examples of displaying a color map or a color bar as a relationship image have been described. In contrast, in the example shown in FIG. 9, the display control function 145 displays an arrow indicating a direction of blood flow as the relationship image 10D on the second region R2.

According to the example shown in FIG. 9, displaying an arrow as the relationship image 10D on the second region R2 enables the user to readily comprehend a regurgitation state based on the direction of the arrow while suppressing a decline in visibility of the mitral valve 3.

### Fourth Modification

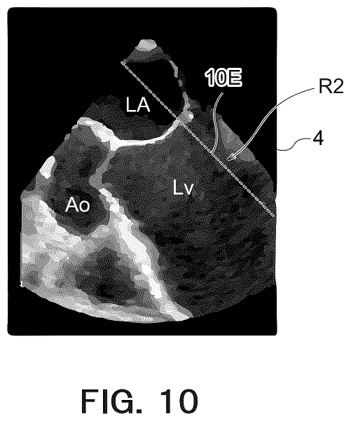

Next, a fourth modification of displaying a straight line as a relationship image will be described with a focus on the differences from the embodiment described above. FIG. 10 is a diagram showing a display step of a relationship image 10E in an operation example of the medical image processing device 1 according to the fourth modification of the embodiment.

In the example shown in FIG. 10, the display control function 145 displays a straight line (for example, a dashed line) indicating a direction of blood flow as the relationship image 10E on the second region R2.

According to the example shown in FIG. 10, displaying a straight line as the relationship image 10E on the second region R2 enables the user to readily comprehend a regurgitation state based on the direction of the straight line while suppressing a decline in visibility of the mitral valve 3.

### Fifth Modification

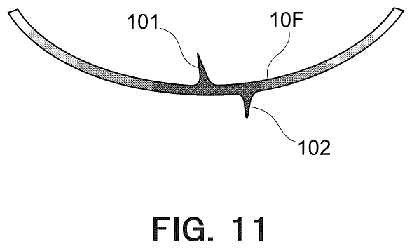

Next, a fifth modification of displaying a relationship image indicating regurgitation information in a depth direction will be described with a focus on the differences from the embodiment described above. FIG. 11 is a diagram showing a display step of a relationship image 10F in an operation example of the medical image processing device 1 according to the fifth modification of the embodiment.

Thus far, examples of displaying a relationship image indicating a relationship of blood flow information in a direction along a display cross-section of the ultrasound image 4 relative to the position of the target site have been described. In contrast, in the example shown in FIG. 11, the display control function 145 displays the relationship image 10F indicating a relationship of blood flow information in a depth direction of the display cross-section of the ultrasound image 4 relative to the position of the target site. In the example shown in FIG. 11, a protrusion amount of a protrusion 101 toward an upper side in the relationship image 10F indicates regurgitation information (for example, a regurgitation amount, a regurgitation velocity, or a regurgitation direction) toward a back side of the display cross-section of the ultrasound image 4 relative to a corresponding position on the target site. In addition, in the example shown in FIG. 11, a protrusion amount of a protrusion 102 toward a lower side in the relationship image 10F indicates regurgitation information toward a front side of the display cross-section of the ultrasound image 4 relative to a corresponding position on the target site. For example, the protrusion amounts (in other words, lengths of spikes) of the protrusions 101 and 102 in FIG. 11 may indicate how much the regurgitation direction is inclined towards the back or the front of the display cross-section.

According to the example shown in FIG. 11, displaying the color bar indicating the relationship of blood flow information in the depth direction of the display cross-section of the ultrasound image 4 relative to the position of the target site on the second region R2 enables the user to readily comprehend a regurgitation state in the depth direction while suppressing a decline in visibility of the mitral valve 3.

### Sixth Modification

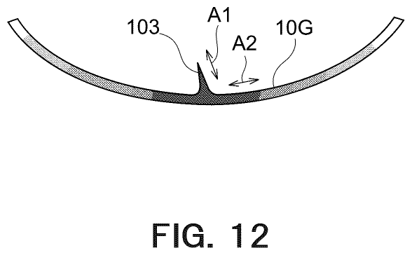

Next, a sixth modification of displaying a relationship image that changes over time will be described with a focus on the differences from the embodiment described above. FIG. 12 is a diagram showing a display step of a relationship image 10G in an operation example of the medical image processing device 1 according to the sixth modification of the embodiment. Thus far, examples of displaying a relationship image indicating a relationship of blood flow information at a predetermined time point (in other words, a cardiac phase) have been described. In contrast, in the example shown in FIG. 12, the display control function 145 displays the relationship image 10G so as to change according to time (in other words, cardiac phase). Specifically, in the example shown in FIG. 12, a protrusion 103 in the relationship image 10G indicates blood flow information in the depth direction of the display cross-section of the ultrasound image 4. As indicated by an arrow A1 in FIG. 12, a protrusion amount and a protrusion direction of the protrusion 103 may change according to the cardiac phase. In addition, as indicated by an arrow A2 in FIG. 12, a position of the protrusion 103 may also change according to the cardiac phase.

The example shown in FIG. 12 enables the user to comprehend a change in the regurgitation state according to time while suppressing a decline in visibility of the mitral valve 3.

### Seventh Modification

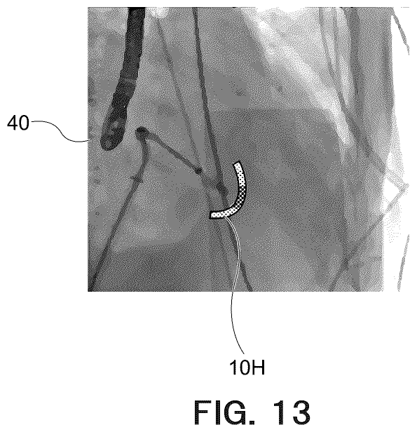

FIG. 13 is a diagram showing a display step of a relationship image 10H in an operation example of the medical image processing device 1 according to a seventh modification of the embodiment. Thus far, examples of displaying a relationship image on the two-dimensional ultrasound image 4 have been described. In contrast, as shown in FIG. 13, the display control function 145 displays the relationship image 10H on an X-ray image 40. In this case, the medical image processing device 1 is an X-ray diagnostic device. Specifically, the blood flow information acquisition function 143 of the X-ray diagnostic device acquires blood flow information of the first region R1 based on an ultrasound image (in other words, a second medical image) of a different type from the X-ray image 40 (in other words, the medical image). The display control function 145 of the X-ray diagnostic device displays the relationship image 10H indicating a relationship of the blood flow information acquired based on the ultrasound image relative to the position of the target site on the target site of the X-ray image 40 or on the second region R2 of the X-ray image 40. Note that the correspondence information generated by the correspondence information generating function 144 is used when displaying the relationship image in a similar manner to the embodiment described above.

Note that the display control function 145 may switchably display the relationship images 10A to 10H of the plurality of aspects described above or may simultaneously display two or more relationship images 10A to 10H. In addition, the display control function 145 may display relationship images on a three-dimensional ultrasound image.

### Eighth Modification

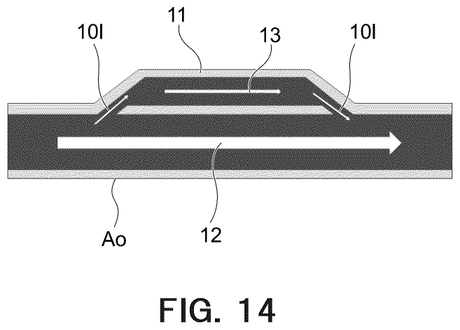

Next, an eighth modification to be applied to an aortic dissection will be described with a focus on the differences from the embodiment described above. FIG. 14 is a diagram showing a display step of a relationship image 10I in an operation example of the medical image processing device 1 according to the eighth modification of the embodiment.

Thus far, examples of displaying a relationship image indicating a relationship of blood flow information relative to the mitral valve 3 as the target site have been described. In contrast, in the example shown in FIG. 14, the target site is the aorta Ao on the medical image. The blood flow information is information indicating a blood flow 13 which branches off from a blood flow 12 in the aorta Ao and which flows into a dissected portion 11 (in other words, a false lumen) of the aorta Ao. In the example shown in FIG. 14, the display control function 145 displays an arrow as the relationship image 10I at positions corresponding to an inflow port to the dissected portion 11 and an outflow port from the dissected portion 11, respectively.

The example shown in FIG. 14 enables the user to readily comprehend a dissection state while suppressing a decline in visibility of the aorta Ao.

### Ninth Modification

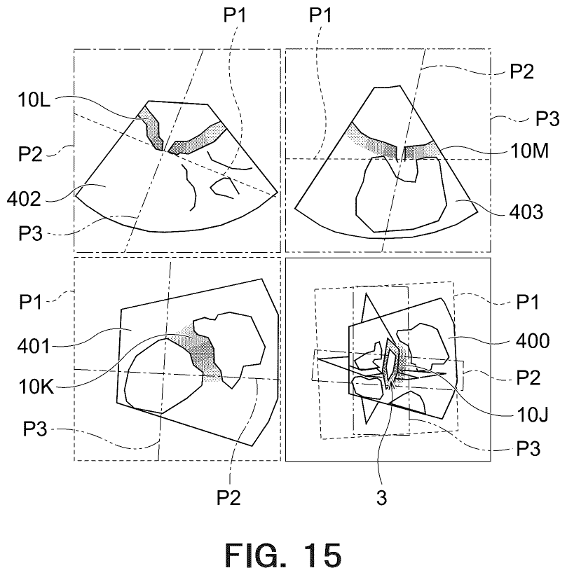

Next, a ninth modification of displaying a three-dimensional relationship image on a three-dimensional medical image will be described with a focus on the differences from the embodiment described above. FIG. 15 is a diagram showing a display step of a relationship image 10J in an operation example of the medical image processing device 1 according to a ninth modification of the embodiment. Thus far, examples of displaying two-dimensional relationship images have been described. In contrast, in the example shown in FIG. 15, the display control function 145 displays a three-dimensional relationship image 10J or, in other words, a color bar along the mitral valve 3 that is an example of the target site on a three-dimensional ultrasound image 400 including the mitral valve 3. The display control function 145 may display the relationship image 10J on the second region described above.

In addition, in the example shown in FIG. 15, the display control function 145 displays two-dimensional ultrasound images 401, 402, and 403 obtained by slicing the three-dimensional ultrasound image 400 at arbitrary cross-sections P1, P2, and P3 using the multiplanar reconstruction (MPR) method. The display control function 145 displays two-dimensional relationship images 10K, 10L, and 10M obtained by slicing the three-dimensional relationship image 10J at the cross-sections P1, P2, and P3 on the two-dimensional ultrasound images 401, 402, and 403.

Displaying a three-dimensional relationship image on a three-dimensional medical image enables a blood flow state in the periphery of the target site to be comprehended in greater detail.

### Tenth Modification

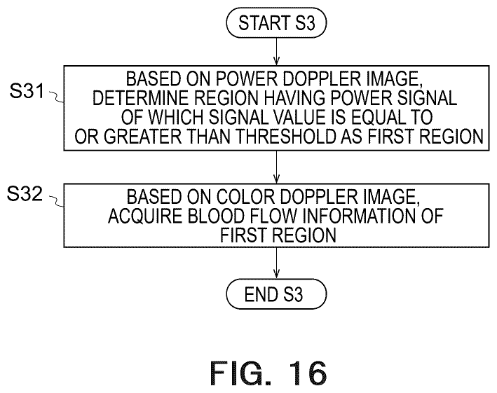

Next, a tenth modification of displaying a relationship image based on both a power Doppler image and a color Doppler image will be described with a focus on the differences from the embodiment described above. FIG. 16 is a flow chart showing an operation example of the medical image processing device 1 according to the embodiment. Thus far, examples of displaying relationship images based on color Doppler images have been described. In contrast, in the example shown in FIG. 16, the display control function 145 displays a relationship image based on both a power Doppler image and a color Doppler image.

Specifically, in the example shown in FIG. 16, when acquiring blood flow information of the first region in the periphery of the target site (step S3), the blood flow information acquisition function 143 first determines a region in the periphery of the target site (for example, the mitral valve) in which a power signal having a signal value equal to or greater than a threshold exists as the first region R1 based on a power Doppler image (step S31). In other words, the blood flow information acquisition function 143 does not determine a region in which a power signal having a signal value less than the threshold exists as the first region R1. In other words, the blood flow information acquisition function 143 excludes regions with a low probability of blood flow from a target acquisition area (first region R1) of blood flow information.

After determining the first region R1, the blood flow information acquisition function 143 acquires blood flow information (direction and velocity) of the first region R1 based on a color Doppler image (step S32). Subsequently, a relationship image is displayed by sequentially performing steps S4 and S5 in FIG. 3.

According to the example shown in FIG. 16, acquiring blood flow information from a region with a power signal (in other words, a region with a high probability of blood flow) prevents information on a region with no blood flow from being reflected onto a relationship image. Accordingly, a relationship image with high reliability can be displayed.

In addition, the display control function 145 may display a relationship image indicating a relationship of power (blood flow information) relative to the position of the target site based on a power Doppler image. For example, the display control function 145 may display a color bar that displays different colors according to the magnitude of power. In addition, the display control function 145 may simultaneously display the color bar (relationship image) that displays different colors according to the magnitude of power and a color bar (relationship image) that displays different colors according to the direction and the velocity of blood flow described above. Alternatively, the display control function 145 may switch between displaying the color bar that displays different colors according to the magnitude of power and the color bar that displays different colors according to the direction and the velocity of blood flow.

As described above, in the embodiment, the position acquisition function 142 acquires a position of a target site on a medical image. In addition, the blood flow information acquisition function 143 acquires blood flow information of a first region R1 in a periphery of the target site. Furthermore, the display control function 145 displays a relationship image indicating a relationship of blood flow information acquired by the blood flow information acquisition function 143 relative to the position of the target site acquired by the position acquisition function 142 on the target site or on a second region R2 that differs from the first region R1.

Accordingly, the blood flow in the periphery of the target site can be visualized while suppressing a decline in visibility of the target site.

In addition, in the embodiment, a relationship includes a direction of blood flow in the blood flow information relative to the position of the target site.

Accordingly, the direction of blood flow relative to the position of the target site can be visualized while suppressing a decline in visibility of the target site.

In addition, in the embodiment, the direction of blood flow is a direction that differs from the normal direction of blood flow.

Accordingly, the direction of blood flow that differs from a normal direction relative to the position of the target site can be visualized while suppressing a decline in visibility of the target site.

In addition, in the embodiment, the direction that differs from the normal direction is a direction opposite to the normal direction.

Accordingly, a regurgitation direction relative to the position of the target site can be visualized while suppressing a decline in visibility of the target site.

In addition, in the embodiment, a relationship further includes at least one of an amount of blood flow in the direction of blood flow and a blood flow velocity in the direction of blood flow.

Accordingly, at least one of an amount of blood flow in the direction of blood flow and a blood flow velocity in the direction of blood flow relative to the position of the target site can be visualized while suppressing a decline in visibility of the target site.

In addition, in the embodiment, the correspondence information generating function 144 generates correspondence information indicating a correspondence relationship between a plurality of positions on the target site and blood flow information. In addition, based on the correspondence information generated by the correspondence information generating function 144, the display control function 145 displays the relationship image so as to indicate a relationship corresponding to each of the plurality of positions.

Accordingly, a relationship image can be appropriately displayed based on correspondence information.

In addition, in the embodiment, the correspondence information generating function 144 generates the correspondence information by associating a plurality of positions with blood flow information existing in a specific direction relative to each of the plurality of positions.

Accordingly, since the correspondence information can be appropriately generated, a relationship image can be more properly displayed.

In addition, in the embodiment, the specific direction includes at least one of a direction of blood flow, a normal direction of a surface of the target site, a travel direction of the procedure device, and a scan line direction of ultrasound in a region of interest.

Accordingly, correspondence information can be more appropriately generated.

In addition, in the embodiment, the target site is a procedure object site.

Accordingly, the blood flow in the periphery of the procedure object site can be visualized while suppressing a decline in visibility of the procedure object site.

In addition, in the embodiment, the blood flow information acquisition function 143 acquires blood flow information in real time during a procedure and acquires blood flow information at a specific time point in the past or acquires blood flow information within a specific time range in the past.

Accordingly, a relationship image can be suitably used to assess blood flow during or after a procedure.

In addition, in the embodiment, the blood flow information acquisition function 143 acquires blood flow information based on at least one of a color Doppler image as a medical image and changes in the shape of the heart or blood vessels in the medical image.

Accordingly, blood flow information can be appropriately acquired.

In addition, in the embodiment, the blood flow information acquisition function 143 updates the acquisition of blood flow information in accordance with progress of procedures.

Accordingly, a relationship image based on the latest blood flow information can be displayed in real time during a procedure.

In addition, in the embodiment, the second region is a region excluding the region through which the procedure device has passed and the region through which the procedure device is to pass.

Accordingly, the blood flow in the periphery of the target site can be visualized while more effectively suppressing a decline in visibility of the target site.

In addition, in the embodiment, the procedure object site is the mitral valve.

Accordingly, the blood flow in the periphery of the mitral valve 3 can be visualized while suppressing a decline in visibility of the mitral valve 3.

In addition, in the embodiment, the relationship image may include at least one of a color map, a color bar, an arrow, and a line segment.

Accordingly, the user can readily comprehend a relationship of blood flow information relative to the target site.

In addition, in the embodiment, the medical image is an ultrasound image, an X-ray image, a CT image, or an MR image.

Accordingly, the versatility of relationship images can be improved.

For example, the term “processor” as used in the description given above means a CPU (Central Processing Unit), a GPU (Graphics Processing Unit), or circuitry such as an Application Specific Integrated Circuit (ASIC) or a Programmable Logic Device (for example, a Simple Programmable Logic Device (SPLD), a Complex Programmable Logic Device (CPLD), or a Field Programmable Gate Array (FPGA)). The processor realizes functions by reading and executing programs stored in storage circuitry. Note that a configuration may be adopted in which a program is directly incorporated into the circuitry of the processor instead of being stored in the storage circuitry. In this case, the processor realizes functions by reading and executing the program incorporated into the circuitry. The processor is not limited to a single circuitry configuration of the processor and a plurality of independent circuitries may be combined to constitute a single processor and the functions may be realized by the single processor. Furthermore, the plurality of constituent elements in FIG. 1 may be integrated into a single processor and the functions may be realized by the single processor.

According to the at least one embodiment described above, the blood flow in the periphery of the target site can be visualized while suppressing a decline in visibility of the target site.

While certain embodiments have been described above, the embodiments have been presented by way of example only and are not intended to limit the scope of the invention. The novel device and method described in the present specification can be implemented in various other modes. In addition, various omissions, substitutions, and modifications can be made to the modes of the device and method described in the present specification to the extent that they do not depart from the gist of the invention. The appended claims and their equivalents are intended to include such modes and modifications that are included in the scope and gist of the invention.

1. A medical image processing device, comprising processing circuitry configured to:
acquire a position of a target site on a medical image;
acquire blood flow information of a first region in a periphery of the target site; and
display an image indicating a relationship of the acquired blood flow information relative to the position of the acquired target site on the target site or on a second region that differs from the first region.

2. The medical image processing device according to claim 1, wherein the relationship includes a direction of blood flow in the blood flow information relative to the position of the target site.

3. The medical image processing device according to claim 2, wherein the direction of the blood flow is a direction that differs from a normal direction of the blood flow.

4. The medical image processing device according to claim 3, wherein the direction that differs from the normal direction is a direction opposite to the normal direction.

5. The medical image processing device according to claim 2, wherein the relationship further includes at least one of an amount of blood flow in the direction of blood flow and a blood flow velocity in the direction of blood flow.

6. The medical image processing device according to claim 1, wherein the processing circuitry is further configured to: generate correspondence information indicating a correspondence relationship between a plurality of positions on the target site or on the second region and the blood flow information; and
display the image so as to indicate the relationship corresponding to each of the plurality of positions based on the generated correspondence information.

7. The medical image processing device according to claim 6, wherein the processing circuitry is configured to generate the correspondence information by associating the plurality of positions with the blood flow information existing in a specific direction relative to each of the plurality of positions.

8. The medical image processing device according to claim 7, wherein the specific direction includes at least one of a direction of blood flow, a normal direction of a surface of the target site, a travel direction of a procedure device, and a scan line direction of ultrasound in a region of interest.

9. The medical image processing device according to claim 1, wherein the target site is a procedure object site.

10. The medical image processing device according to claim 1, wherein the processing circuitry is configured to acquire blood flow information in real time during a procedure, acquire blood flow information at a specific time point in the past, or acquire blood flow information within a specific time range in the past.

11. The medical image processing device according to claim 1, wherein the processing circuitry is configured to acquire the blood flow information based on at least one of a color Doppler image as the medical image and changes in the shape of the heart or blood vessels in the medical image.

12. The medical image processing device according to claim 1, wherein the processing circuitry is configured to update the acquisition of the blood flow information in accordance with progress of a procedure.

13. The medical image processing device according to claim 1, wherein the second region is a region excluding a region through which a procedure device has passed and a region through which the procedure device is to pass.

14. The medical image processing device according to claim 9, wherein the procedure object site is the mitral valve.

15. The medical image processing device according to claim 1, wherein the image includes at least one of a color map, a color bar, an arrow, and a line segment.

16. The medical image processing device according to claim 1, wherein the medical image is an ultrasound image, an X-ray image, a CT image, or an MR image.

17. The medical image processing device according to claim 1, wherein the processing circuitry is configured to acquire blood flow information of the first region based on a second medical image of a different type from the medical image, and
display an image indicating a relationship of the blood flow information acquired based on the second medical image relative to the position of the target site on the target site of the medical image or on the second region of the medical image.

18. The medical image processing device according to claim 17, wherein the medical image is an X-ray image and the second medical image is an ultrasound image.
